(19) United States
(12) Patent Application Publication
(10) Pub. No.: US 2014/0235725 A1
(43) Pub. Date: Aug. 21, 2014

(54) PRECONFIGURED GEL WITH INFUSED DRUG FOR ULTRASOUND

(71) Applicant: G. Chad MORGAN, Chattanooga, TN (US)

(72) Inventor: G. Chad MORGAN, Chattanooga, TN (US)

(21) Appl. No.: 14/180,963

(22) Filed: Feb. 14, 2014

Related U.S. Application Data
(60) Provisional application No. 61/765,392, filed on Feb. 15, 2013.
(60) Provisional application No. 61/765,376, filed on Feb. 15, 2013.

(51) Int. Cl. A61K 9/06; A61K 41/00; A61K 31/167

(52) U.S. Cl. CPC A61K 9/06; A61K 31/167; A61K 41/0047
USPC 514/626

(57) ABSTRACT

Embodiments associated with an ultrasound conductive medium are described. In one embodiment, an ultrasound conductive medium is formed into a preconfigured shape, wherein the ultrasound conductive medium includes a composition combined with a drug.

### CROSS REFERENCE TO RELATED APPLICATIONS

This patent disclosure claims the benefit of U.S. Provisional Patent Application Ser. No. 61/765,392 entitled “Preconfigured gel with infused drug for ultrasound” filed on Feb. 15, 2013, and U.S. Provisional Patent Application Ser. No. 61/765,376 entitled “Preconfigured gel for ultrasound” filed on Feb. 15, 2013, which are both hereby wholly incorporated by reference in their entirety.

### BACKGROUND

Ultrasound devices operate with frequencies from 20 kHz up to several gigahertz. Ultrasound is a method of stimulating the tissue beneath the skin's surface using very high frequency sound waves and/or to provide imaging of internal structures.

Ultrasound is applied using a device that includes a transducer or applicator that is in contact with a patient's skin. Gel is used on all surfaces of the device's head to reduce friction and assist transmission of the ultrasonic waves. The gel is typically squeezed out of a bottle and spread over the patient's skin. The gel is difficult to contain within a desired area of the skin and the thickness of the gel cannot be controlled. Lack of consistent and desired thickness of the gel leads to a less than optimal ultrasound application. When the ultrasound procedure is completed, the patient is required to wipe off the gel from the patient's skin. Typically, the gel is not completely removed and the cleaning process is uncomfortable.

### SUMMARY

In one embodiment of the disclosure, an ultrasound conductive medium is disclosed that is formed into a preconfigured shape, wherein the ultrasound conductive medium includes a composition combined with a drug. In another aspect, the ultrasound conductive medium is in a solidified state that maintains the preconfigured shape. In another aspect, the drug includes an anesthetic. In another aspect, the combined composition of the ultrasound conductive medium includes 1% to 10% Lidocaine.

In another embodiment of the disclosure, the ultrasound conductive medium includes a preconfigured shape that is a self-contained piece of gel that can be individually handled. In another aspect, the preconfigured shape is a cap-like structure.

In another embodiment of the disclosure, a gel is disclosed that is preconfigured into a solid form having a shape defined to correspond to a head of an ultrasound device, wherein the gel is infused with a drug. In one embodiment, the solid form of the gel is generally a pad shape configured to fit into a cavity of the head of the ultrasound device. In one embodiment, the drug includes a composition that comprises Lidocaine. In another embodiment, the drug is an anesthetic

In another embodiment of the disclosure, the solid form of the gel is a combination of an ultrasound conductive medium and the drug in a solidified state. In another embodiment, the gel is configured to deliver the drug to a patient in response to ultrasound energy being applied to the gel.

In another embodiment, the solid form of the gel is configured to provide at least two functions of (1) acting as a coupling agent for the ultrasound device, and (2) providing a dose of the drug during an ultrasound procedure.

In another aspect, the solid form of the gel is a solidified state where the gel ranges from a soft and flexible object to a rigid object.

In another embodiment of the disclosure, a method is disclosed that comprises: providing a composition of ultrasound conductive medium; combining the ultrasound conductive medium with a drug; forming a selected amount of the combined composition into a shape; and processing the selected amount to create a solid form of the shape.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, or other shapes) in the figures represent one embodiment of the boundaries. In some embodiments one element may be designed as multiple elements or that multiple elements may be designed as one element. In some embodiments, an element shown as an internal component of another element may be implemented as an external component and vice versa. Furthermore, elements may not be drawn to scale.

FIGS. 1A and 1B illustrate one embodiment of a preconfigured gel from a top view and side view, respectively.

FIG. 1C illustrates another embodiment of the preconfigured gel from a side view.

FIG. 1D illustrates a 3-dimensional image of the preconfigured gel.

FIG. 2A illustrates one embodiment of an ultrasound device shown with components unassembled.

FIG. 2B illustrates the ultrasound device of FIG. 2A in an assembled view.

FIGS. 3A-3D illustrate various views of one embodiment of a diaphragm.

FIG. 4A shows the preconfigured gel 140 being inserted into a cavity of the diaphragm.

FIG. 4B shows the preconfigured gel 140 in an inserted state in the cavity of the diaphragm.

FIG. 5A shows one embodiment of a perspective view of a preconfigured gel cover;

FIG. 5B shows a cross-section view of FIG. 5A.

FIG. 6 shows one embodiment of a method associated with creating an ultrasound conductive medium combined with a drug.

### DETAILED DESCRIPTION

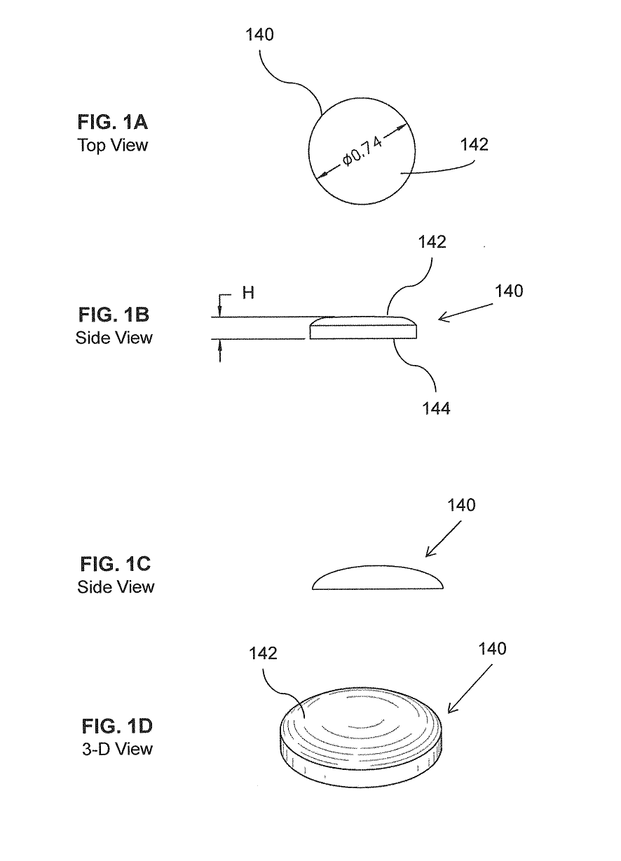

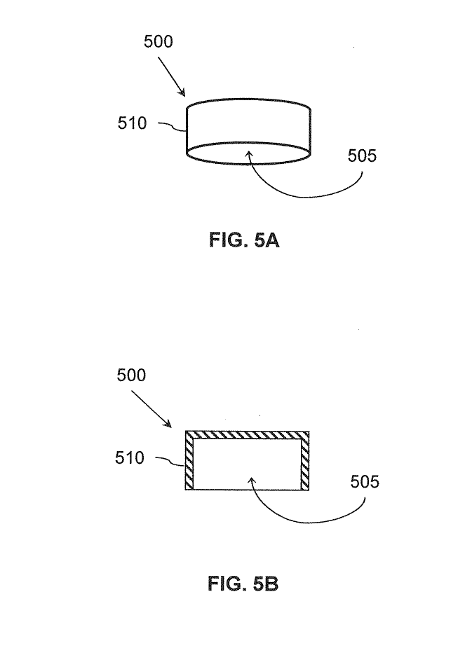

Embodiments of preconfigured gel are disclosed herein that are used for ultrasound devices. In one embodiment, ultrasound gel is preconfigured into a predefined shape (herein also referred to as a “gel shot”, which is an individual piece of gel). In general, gel refers to an ultrasound conductive medium. As will be described with reference to FIGS. 1-3, the predefined shape is made to correspond to a recessed cavity of an ultrasound device in which the preconfigured gel is inserted. In one embodiment, the preconfigured gel is infused with a drug to be delivered during an ultrasound procedure (e.g., transdermal delivery). In one embodiment, the drug is Lidocaine or other anesthetic. In another embodiment, as will be described with reference to FIG. 5A, gel is preconfigured into a defined shape to cover the head of an ultrasound device (herein also referred to as a “gel cover” or cap).

In one embodiment, the preconfigured gel (gel shot or gel cover) is formed from an aqueous material that functions as a conductive medium for ultrasound energy. The aqueous material is for example a liquid or jelly-like substance that is molded into a desired shape and processed to retain its shape as a solid form (e.g., substance is solidified via curing or other process). In one embodiment, after being solidified the preconfigured gel is a soft and flexible object, semi-rigid and flexible object/structure, or other solid state. The gel shot or gel cover is formed to retain its shape and have memory which are properties of the gel composition used to create the preconfigured piece of gel. Thus the preconfigured gel provides a consistent amount and thickness of gel for an ultrasound procedure. The gel shot or gel cover is formed in advance so that it is ready for use with an ultrasound device. After being used in an ultrasound procedure, the preconfigured gel is removed from the ultrasound device and disposed. A new piece of preconfigured gel can then be used for the next procedure.

With reference to FIG. 1A and 1B, one embodiment of preconfigured gel 140 is illustrated. FIG. 1A shows a top view of the gel 140 and FIG. 1B shows a side view. FIG. 1C illustrates a side view of another embodiment of the gel 140 with a different shape. FIG. 1D illustrates a 3-dimensional image of the preconfigured gel 140 where the gel 140 represents the shape of FIGS. 1A and 1B.

In one embodiment, the preconfigured gel 140 is formed with a circular top surface 142 and bottom surface 144 and has a general configuration of a disk/pad. The preconfigured gel 140 includes a thickness or height H. In one embodiment, the height H of the gel 140 is about 0.15 inches and the diameter is about 0.74 inches. In another embodiment, the diameter may be about 1.17 inches for use with ultrasound devices that have a larger circular head. Of course, other dimensions may be implemented.

The shape and size of the preconfigured gel 140 may be defined to correspond to a gel receiving cavity of an ultrasound device in which the gel 140 is to function with. Of course, the gel 140 may be implemented in other shapes and sizes (e.g., 3-dimensional oval or polygon, other shapes with curved sides, flat sides, rectangular shape, and so on). The top surface 142 may be flat (see FIG. 1B) or curved (see FIG. 1C) in order to provide good contact with a patient's skin. In other embodiments, the preconfigured gel 140 may be formed with irregular shapes such as having the top surface 142 that is larger than the bottom surface 144 (e.g., mushroom shape). In other embodiments, the gel cover may be formed in various shapes and sizes to conform to the applicable ultrasound device head.

### DRUG INFUSED EMBODIMENT

In one embodiment, the preconfigured gel 140 is combined/infused with a drug. The drug infused gel is configured to deliver the drug during an ultrasound procedure utilizing phonophoresis. For example, when ultrasound energy is applied to the preconfigured gel 140, the ultrasound energy assists to drive and deliver the drug from the gel and into the skin of a patient (e.g., transdermal delivery). Thus, the preconfigured gel 140 provides at least two functions of (1) acting as a coupling agent for the ultrasound device, and (2) providing a dose of a drug during the ultrasound procedure.

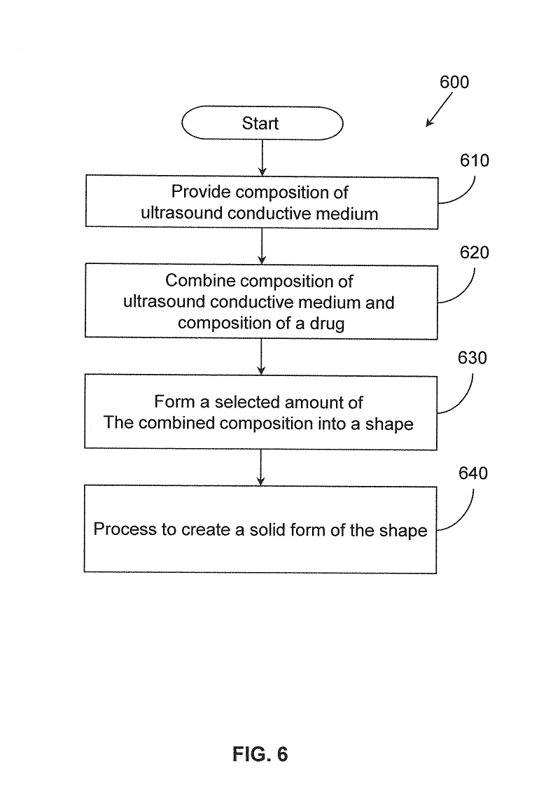

In general, the drug (with or without other additives) is combined with the composition of the gel before the gel is formed into a preconfigured shape. For example, with reference to FIG. 6, one embodiment of a method 600 for creating the preconfigured gel is shown. At 610, a composition of an ultrasound conductive medium is provided, for example, in a container. The composition may have different formulas of ingredients based on the type of ultrasound conductive medium desired (hereinafter referred to as gel composition). At this point, the gel composition is generally in a flowable form (nonsolid). At 620, a drug composition/formula is added and combined with the gel composition.

Combining the gel composition with the drug composition may be performed in a variety of ways such as where the drug is wholly or partially infused into, mixed into, absorbed into, penetrated into, or otherwise incorporated into the gel composition. At 630, a selected amount of the combined substance is then formed or molded into a desired shape. This may involve using molds where multiple molds can be used to create multiple pieces of the preconfigured gel at a time. For example, a tray of patterned shapes can be used where the gel composition is inserted into each shape. Other processes may be used such as injection molding, 3-D printing, and so on. At 640, the combined gel/drug composition is processed to solidify the gel to a desired extent so that its shape is set (e.g., curing, heating, cooling, or other process).

In another embodiment, the drug composition may be combined or added to the gel after the gel is solidified into a shape. For example, the drug composition may be impregnated into, infused into, coated onto, or otherwise interpenetrated into the preconfigured gel. Of course, other processes can be used to combine the gel and the drug compositions.

In general, the gel and drug combination is formed into a self-contained piece of gel that can be individually handled from other pieces of gel and applied to or used with an ultrasound device. In one embodiment, individual pieces of the preconfigured gel 140 may be packaged in a group of preconfigured gel pieces that can be removed and used individually during ultrasound procedures. In other embodiments, each preconfigured gel 140 is packaged individually to ensure each gel piece is sterile.

In one embodiment, the preconfigured gel 140 is infused with a selected percentage of Lidocaine (e.g., 1% to 10%). In other embodiments, other types of medicines or anesthetics can be infused/combined with an ultrasound conductive medium to create a solid piece of preconfigured gel 140. By combining the drug within the preconfigured gel 140 (which acts as a coupling agent for the ultrasound), drug penetration may be improved or maximized through the tissue of a patient by the use of phonophoresis. Phonophoresis is the use of ultrasound to enhance the delivery of topically applied drugs.

During an ultrasound procedure, medicines contained within or under the ultrasound gel 140 are pushed by the sound waves of the ultrasound and driven below the skin surface of a patient. Phonophoresis allows the medication to penetrate the body at a much deeper level than topical creams and lontophoresis. Thus in other embodiments, the drug may be any medicine or agent that can be driven or activated by ultrasound from the preconfigured gel 140 and be caused to penetrate into the skin of a patient.

### ULTRASOUND DEVICE EMBODIMENT

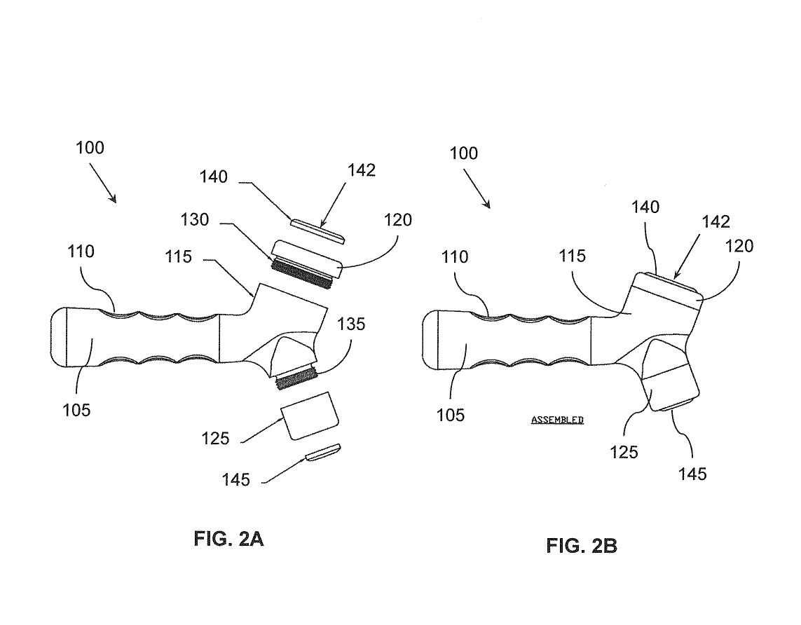

With reference to FIGS. 2A and 2B, in one embodiment, an ultrasound device 100 described herein includes a diaphragm configured with a recessed cavity for receiving and containing the preconfigured gel shot 140. In another embodiment, the diaphragm is configured as a replaceable head for an ultrasound device where the diaphragm includes an ultrasound transducer (e.g., piezoelectric crystal) and a cavity for containing the preconfigured gel 140.

FIG. 2A illustrates the ultrasound device 100 in a partially unassembled state and FIG. 2B shows the device 100 in an assembled state. The device 100 is configured as a hand-held device including an elongated handle 105 that may include one or more finger grips 110 (e.g., indentations, ridges, and so on). The handle 105 is connected to a head 115 that includes one or more sides that connect to a diaphragm. In the illustrated embodiment, the head 115 includes two sides where the first side includes diaphragm 120 and the second side includes diaphragm 125. In other embodiments, the ultrasound device/probe has only one head.

In general as discussed herein, the diaphragm 120, 125 is the component that is formed or connected as part of the head (sometimes referred to as the nose) of an ultrasound device/probe. The diaphragm may also be an acoustic member or acoustic lens such that ultrasound energy generated from a transducer is directed and transmitted through the acoustic lens/diaphragm, and in imaging probes, echo signals are received from a subject through the acoustic lens/diaphragm.

In one embodiment, diaphragm 120 is configured with a connector 130 that is threaded to insert and connect with a corresponding threaded socket in the head 115. Similarly, the second side of the head 115 may include a connector 135 that is threaded for connecting with a threaded socket within the diaphragm 125. In another embodiment, the connectors 130 and 135 may be configured as a quick-connect/disconnect device so that the diaphragms 120 and 125 can be connected by pushing and snapping into place or disconnected by pulling off with a small amount of force. In another embodiment, the diaphragm 120 may be configured to slide on/off and connect by friction.

Thus, in one embodiment, the diaphragms 120 or 125 are configured as replaceable components that can be removed and attached to other devices (e.g., attachable and detachable). Being replaceable allows for different sized diaphragms to be connected to the head 115 so that the same device 100 can be configurable with different sized diaphragms and different sized transducers that may be attached within the diaphragm. In another embodiment, the head 115 and diaphragm 125 may be integral with each other (see example in FIG. 5).

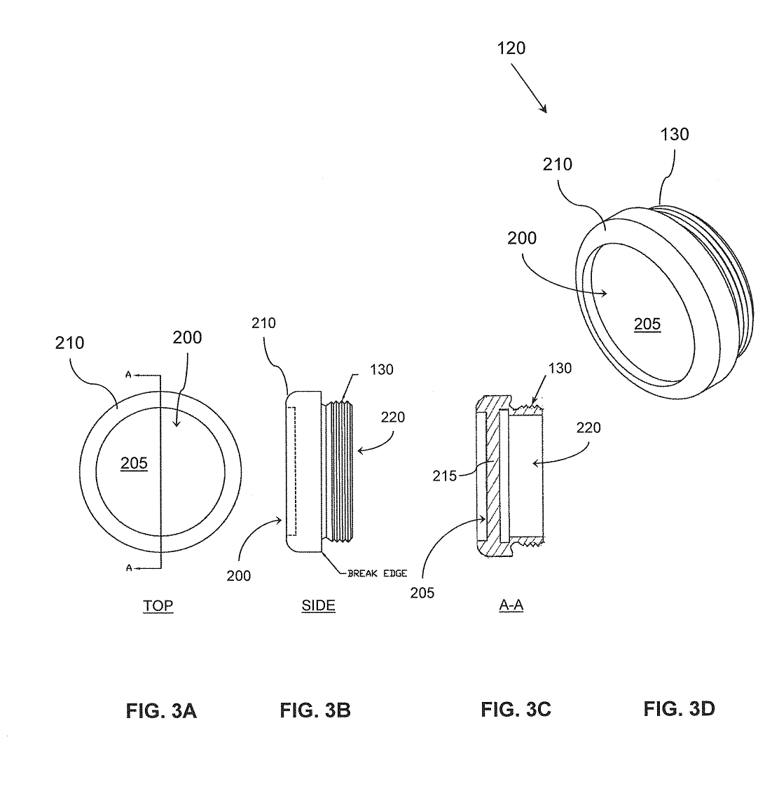

With reference to diaphragm 120, the diaphragm includes a recessed cavity/receptacle (shown in FIG. 3D) that is configured to receive and contain/hold a conductive medium (preconfigured gel 140) used during an ultrasound scan. In one embodiment, the conductive medium 140 is a portion of gel as described previously that is preconfigured to fit into the cavity, as seen in FIG. 2B where the preconfigured gel 140 is inserted into the diaphragm 120. Likewise, diaphragm 125 includes a cavity to receive a conductive medium 145 when used during a scan. The diaphragm 120 is explained in more detail with reference to FIG. 2.

With reference to FIGS. 3A-3D, one embodiment of diaphragm 120 is shown in a Top View FIG. 3A, Right Side View FIG. 3B, Cross-Section View FIG. 3C through A-A, and a Perspective View FIG. 3D.

In one embodiment, the diaphragm 120 a housing formed from metal, metallic, or other conductive material that functions with ultrasound energy (e.g., plastic or other acoustic conducting material). The diaphragm 120 includes a cavity/receptacle 200 that is configured to receive a conductive medium. For example, the cavity 200 is a gel receiving cavity (e.g., a receptacle in which preconfigured gel is inserted for an ultrasound procedure). The cavity 205 is defined by inside surface 205 and a sidewall 210. The surface 205 is generally a circular shape but other shapes may be used.

In one embodiment, the sidewall 210 is a continuous edge or rim around the perimeter of the surface 205. In another embodiment, the sidewall 205 may include one or more notches (not shown). A notch may be used to remove a preconfigured piece of gel from within the cavity 200 by inserting a finger in the notch to access the gel within the cavity. In another embodiment, the sidewall 205 may be perforated or be configured as two or more portions such as prongs that can hold a piece of solid gel (e.g., preconfigured gel 140 shown in FIG. 1A and 1B).

The cavity 205 is configured as a containment area for receiving a conductive preconfigured gel (e.g., a gel shot). In one embodiment as explained previously, the conductive medium is configured to correspond to the shape of the cavity 205. Preconfigured gel 140 can be inserted into the cavity 200 where the gel is held in place by at least surface tension with the surface 205 and/or friction with the inside surface of the sidewall 210. In this manner, the diaphragm 120 self-contains the gel to be used during a scan. A gel cover can be inserted over the head or diaphragm of an ultrasound device and held in place by at least surface tension and/or friction. When a scan is complete, the preconfigured gel is simply removed and another piece of gel can be inserted for a subsequent scan.

In one embodiment, in the inside of the diaphragm 120, the diaphragm 120 includes a transducer (e.g., piezoelectric crystal) (not shown) for generating ultrasound waves. The transducer is connected to the diaphragm within the threaded shaft 130 and secured against inside surface of the surface 205 (e.g., on the back side of surface 205).

FIG. 3C illustrates a cross-section view through A-A. The various dimensions shown (e.g., in inches) are only exemplary of one embodiment. It is not intended to limit the construction of the diaphragm shown since the diaphragm can be formed with different shapes and sizes.

### DEVICE COMPONENTS

With reference again to FIG. 2A, the handle 105 and head 115 are formed from a housing that contains one more components (not shown) configured to generate and detect ultrasound energy. In one embodiment, the device 100 includes an energy generating module operative to generate a driving signal that can be transformed into ultrasonic energy. The energy generating module includes a local power source or receives power from a remote source via a power cord, an oscillator, and a driver component. The portable ultrasound device 100 also includes an ultrasound transducer having a piezoelectric or electric component. The ultrasound transducer is operative to receive the driving signal from the energy generating module and transforms the driving signal into ultrasonic energy. There are many different types of internal components that can be used to implement the ultrasound device. Since they are not the focus of the present disclosure, they are not described in detail.

In another embodiment, the device 100 may include an internal memory for storing ultrasound data collected by the device 100. The device 100 may include an interface for communicating the data from the memory to a remote device. The device 100 can be configured to communicate the data via a wire connection and/or a wireless connection.

### PRECONFIGURED GEL EMBODIMENT

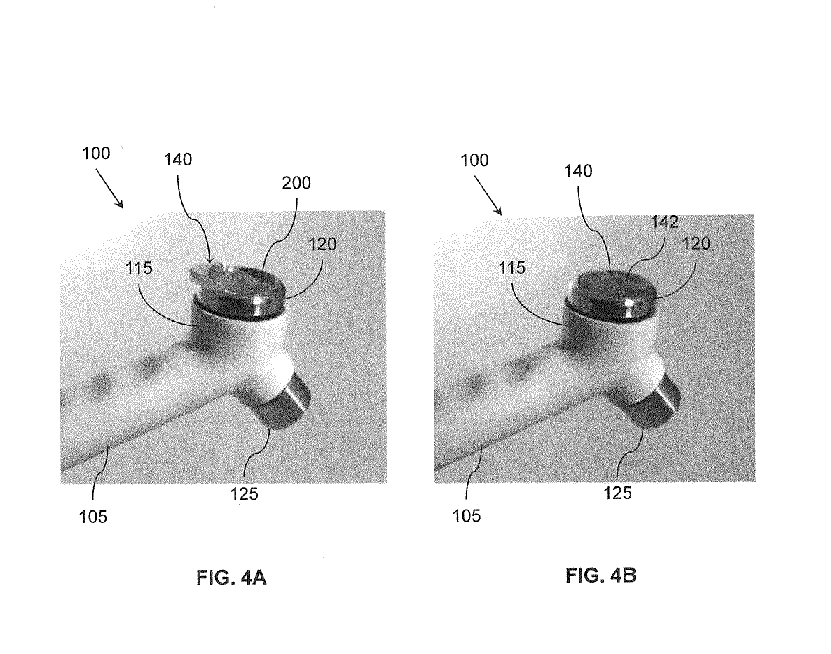

With reference to FIG. 4A, in another embodiment, the preconfigured gel 140 is a conductive medium for use with therapeutic ultrasounds, imaging ultrasounds and/or electrotherapy devices. As previously described, the conductive medium 140 is preconfigured in a pad or disk shape that fits into and is contained within the cavity 200 of the diaphragm 120 or head 115 of the ultrasound device. The medium 140 is solidified into the desired shape. In one embodiment, the preconfigured gel 140 is configured to fit into and generally correspond to the shape of the cavity 200 of a desired ultrasound device. In general, being preconfigured refers to the gel being formed in advance into a solidified shape with a defined size, defined shape, and defined amount of medium for use during an ultrasound procedure. In different embodiments, the preconfigured gel can be solidified to different degrees or ranges as desired such as being a soft and flexible object, being a rigid object, or any state in between.

With reference again to FIG. 4A, the preconfigured gel 140 is shown being inserted into the cavity 200 of the diaphragm 120. FIG. 4B shows the gel shot 140 in an inserted state in the cavity 200 of the diaphragm 120. Once inserted into the cavity 200, an exterior surface (e.g., top surface 142) of the gel 140 extends out from the diaphragm so that the gel 140 contacts the skin of a patient (see FIG. 2B) to minimize air pockets between the skin and the transducer (not shown) in the diaphragm 120. In one embodiment, the gel 140 functions as a standoff between a patient's skin and the diaphragm 120 or covering the diaphragm. Thus during a scan, the preconfigured gel 140 moves with the ultrasonic device by being held within the diaphragm 120. In one embodiment, the gel is maintained within the cavity 200 by friction between the gel and the surfaces of the diaphragm. Spreading liquid gel on a patient is eliminated by using preconfigured gel.

As one example of dimensions, which is not intended to be limiting, the height H of the preconfigured gel 140 is constructed to be slightly greater than the height of the sidewall 210 that defines the cavity 200 (see FIG. 3B and 3C). For example, the height H of the preconfigured gel 140 may be about 0.15 inches and the height of the sidewall 210 may be about 0.092 inches. Of course, other relationships of size can be implemented.

Air and other gases may impede sound waves. In one embodiment, the preconfigured gel 140 is a solid piece of gel yet flexible. The gel 140 prevents the formation of air bubbles between the transducer and the patient's skin and helps conduct sound waves from the transducer into the patient's body.

### PRECONFIGURED GEL COVER EMBODIMENT

In another embodiment, with reference to FIGS. 5A and 5B, gel combined with a drug, which is solidified to a desired extent, is configured to operate with a flat ultrasound device (e.g., no gel cavity in the head/diaphragm). FIG. 5A shows one embodiment of a perspective view of a preconfigured gel and FIG. 5B shows a cross-section view of FIG. 5A. The preconfigured gel is shaped as a gel cover 500 that can be attached like a cap over the head of an ultrasound device (e.g., a cap, sleeve, or other covering type shape). Of course, other shapes can be used based on the shape and configuration of an ultrasound head.

In one example, the gel cover 500 is configured with a cavity 505 to receive the head/diaphragm. The gel cover 500 includes sides 510 that are configured to surround the head/diaphragm of an ultrasound device. In general, this is a reverse configuration as shown in FIG. 4A. Instead of the gel inserting into the cavity of the diaphragm, the diaphragm/head is inserted into the gel cover 500.

In one embodiment, the gel cover 500 may be a configured similar to a shower-cap design (e.g., cap-like structure, functions as a cap) with flexible side walls that can move and stretch to surround and cover the diaphragm/head of a device when attached. In another embodiment, the sidewalls 510 may be configured to fit around the diaphragm (e.g., see FIG. 5B, generally a U-shaped cross-section) so that when the ultrasound head is inserted into the cavity 505 and moved by an operator, the gel cover 500 is moved/pushed along with the device to slide along a patient. In other embodiments, the sidewalls 510 may be much longer to fit a long and narrow ultrasound probe (e.g., stick-like probe). Of course, other shapes can be implemented based on the shape of the ultrasound device.

For an ultrasound procedure, the gel cover 500 is attached over and covers the head of the ultrasound device. The gel cover 500 moves with the ultrasonic device since the gel cover 500 is attached to and holds onto the device with its side walls. Thus the gel cover 500 provides a consistent about of conductive medium between a patient and the ultrasound device and delivers a dosage of a drug.

### DEFINITIONS

The following includes definitions of selected terms employed herein.

The definitions include various examples and/or forms of components that fall within the scope of a term and that may be used for implementation. The examples are not intended to be limiting. Both singular and plural forms of terms may be within the definitions.

The term “conductive medium” is used to refer to a substance that is used during an ultrasound procedure that assists in coupling the ultrasound device/probe head or applicator tip to a subject (e.g., the skin of a patient or other surface) and conducts ultrasound energy. Typically, the conductive medium is ultrasound gel but other substances can be used such as shampoo, hairstyling gel, hand lotion, hand sanitizer, liquid dishwashing detergent, olive oil (or other oil based substances), or other substance that is appropriate to function with an ultrasound device. Many substances can form gels when a suitable thickener or gelling agent is added to their formula to change the viscosity. These substances may be preconfigured into a solidified state as an individual piece of conductive medium (e.g., solidified state may be any state in which the gel holds its shape and does not flow). References to the term “gel” is intended to refer to any of these conductive media that is appropriate for an ultrasound procedure.

References to “one embodiment”, “an embodiment”, “one example”, “an example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

While example systems, methods, and so on have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and so on described herein. Therefore, the disclosure is not limited to the specific details, the representative apparatus, and illustrative examples shown and described. Thus, this application is intended to embrace alterations, modifications, and variations that fall within the scope of the appended claims.

To the extent that the term “includes” or “including” is employed in the detailed description or the claims, it is intended to be inclusive in a manner similar to the term “comprising” as that term is interpreted when employed as a transitional word in a claim.

To the extent that the term “or” is used in the detailed description or claims (e.g., A or B) it is intended to mean “A or B or both”. When the applicants intend to indicate “only A or B but not both” then the phrase “only A or B but not both” will be used. Thus, use of the term “or” herein is the inclusive, and not the exclusive use.

What is claimed is:

1. An ultrasound conductive medium formed into a preconfigured shape, wherein the ultrasound conductive medium includes a composition combined with a drug.

2. The ultrasound conductive medium of claim 1, wherein the ultrasound conductive medium is in a solidified state that maintains the preconfigured shape.

3. The ultrasound conductive medium of claim 1, wherein the drug includes an anesthetic.

4. The ultrasound conductive medium of claim 1, wherein the combined composition of the ultrasound conductive medium includes 1% to 10% Lidocaine.

5. The ultrasound conductive medium of claim 1, wherein the preconfigured shape is a self-contained piece of gel that can be individually handled.

6. The ultrasound conductive medium of claim 1, wherein the preconfigured shape is a cap-like structure.

7. The ultrasound conductive medium of claim 1, wherein the preconfigured shape of the ultrasound conductive medium is configured to provide at least two functions of (1) acting as a coupling agent for an ultrasound device, and (2) providing a dose of the drug during an ultrasound procedure.

8. A gel preconfigured into a solid form having a shape defined to correspond to a head of an ultrasound device, wherein the gel is infused with a drug.

9. The gel of claim 8, wherein the solid form of the gel is generally a pad shape configured to fit into a cavity of the head of the ultrasound device.

10. The gel of claim 8, wherein the drug includes a composition that comprises Lidocaine.

11. The gel of claim 8, wherein the solid form of the gel is a combination of an ultrasound conductive medium and the drug in a solidified state.

12. The gel of claim 8, wherein the drug is an anesthetic.

13. The gel of claim 8, wherein the gel is configured to deliver the drug to a patient in response to ultrasound energy being applied to the gel.

14. The gel of claim 8, wherein the solid form of the gel is configured to provide at least two functions of (1) acting as a coupling agent for the ultrasound device, and (2) providing a dose of the drug during an ultrasound procedure.

15. The gel of claim 8, wherein the solid form of the gel is a solidified state where the gel ranges from a soft and flexible object to a rigid object.

16. A method comprising:
providing a composition of ultrasound conductive medium;
combining the ultrasound conductive medium with a drug;
forming a selected amount of the combined composition into a shape; and
processing the selected amount to create a solid form of the shape.
